(19) United States
(12) Patent Application Publication
(10) Pub. No.: US 2021/0393198 A1
(43) Pub. Date: Dec. 23, 2021

(54) METHODS FOR SETTING UP A CONTROLLER OF AN ORTHOPEDIC DEVICE AND SYSTEM FOR CARRYING OUT THE METHOD

(71) Applicant: Otto Bock Healthcare Products GmbH, Wien (AT)

(72) Inventors: Peter GOEBEL, Wien (AT); Günther WALTER, Vösendorf (AT); Markus SCHACHINGER, Wien (AT); Sigrid GERGER, Wien (AT); Sebastian AMSUESS, Wien (AT)

(73) Assignee: Otto Bock Healthcare Products GmbH, Wien (AT)

(21) Appl. No.: 17/288,438

(22) PCT Filed: Oct. 23, 2019
(86) PCT No.: PCT/EP2019/078897

§ 371 Date: Apr. 23, 2021

(30) Foreign Application Priority Data
Oct. 26, 2018 (DE) 10 2018 126 788.6

(51) Int. Cl. A61B 5/00; A61B 5/389; A61F 2/54; A61F 2/72

(52) U.S. Cl. CPC A61B 5/4851; A61B 5/389; A61B 5/6811; A61B 5/7475; A61B 2560/02; A61F 2/72; A61F 2002/543; A61B 2505/09; A61F 2/54

(57) ABSTRACT

The invention relates to a method for setting up a controller of an orthopedic device with at least one motor drive, which is applied to a body part of the patient and connected to sensors that record control signals of the patient, said method including the following steps: outputting an optical, acoustic and/or tactile representation of an actuation of a limb as a request for the patient to carry out said actuation; detecting control signals that are produced by the patient as a voluntary reaction following the request, assigning the detected control signals to the implemented actuation and to a function in which the at least one motor drive is activated, deactivated or reversed in terms of its direction of rotation, and outputting the detected control signals and/or the function following the assignment to the respective function.

The invention relates to a method for setting up a controller of an orthopedic device comprising at least one motor drive, wherein the orthopedic device is placed against a body part of a patient and connected to sensors that record control signals of a patient. The invention also relates to a system for carrying out such a method.

Prostheses serve to replace the shape and/or function of a missing limb or a body part. In addition to prostheses which only replace the shape of the limbs no longer present or of the body part no longer present, there are prostheses that have a functionality. A prosthesis should frequently replace both the shape and the functionality as faithfully as possible.

By way of example, prostheses that replace a function include gripping devices, by means of which articles can be gripped and held. The actuation of such a gripping device, which may be embodied as a so-called hook, for example, can be implemented by way of a Bowden cable mechanism or in motor driven fashion. There are motor driven prosthetic hands comprising fingers that replicate both the shape and the function of a natural hand and that are controlled by way of myoelectric signals such that different gripping movements and orientations of the prosthetic hand can be adopted on the basis of myoelectric signals.

Prostheses for lower extremities can be embodied as passive prostheses which have no adjustment devices. Dampers, which influence movements between two components connected in articulated fashion, can be provided but no provision is made for an adjustment thereof. Moreover, there are prostheses for lower extremities in which damper settings can be adjusted by way of motor drives on the basis of captured sensor data, for example accelerations, forces, or angles. Likewise, active prostheses are known, which comprise motors that assist or independently bring about flexion and/or extension. These motors are activated or deactivated by way of a controller. Myoelectric control, within the scope of which muscle contractions are recorded and used as control signals, is also known.

In addition to prostheses, corresponding orthoses are known, which are placed against present limbs or body parts and which influence or assist movements or influence or maintain assignments of body parts. Orthoses can also be provided with brakes, dampers, or motor drives in order to change resistances or assist or carry out movements.

To be able to control orthopedic devices such as orthoses or prostheses, use is made of sensors that capture signals produced by the user of the orthopedic device or by the use of the orthopedic device. These can be a multiplicity of signals, for example myoelectric signals or state, load, and/or movement signals. To be able to suitably control the orthopedic device by way of myoelectric signals, the patients or users of the orthopedic device must learn certain contraction patterns which are assigned to the respective functions. Conventionally, a program in which a certain sequence of control signals with different numbers of signal pulses, different signal strengths, and/or different signal durations is defined is stored in the respective controller of the orthopedic device, for example the orthosis or prosthesis. From multiple tensing of a certain muscle or a certain residual muscle at a certain frequency and with a certain intensity, for example, it is possible to recognize that a certain program should be carried out. Thereupon, a drive is activated, for example to alter a damper device in respect of the resistance or in order to displace two components of an orthopedic device with respect to one another. By way of example, in the case of a prosthetic hand, this allows opening or closing of the hand, the rotation of the wrist, or an extension or flexion of the prosthetic hand to be carried out. In the case of a driven prosthetic foot, it is possible to bring about or assist plantarflexion and dorsiflexion. Particularly in the case of orthopedic devices with a multiplicity of functions, for example a prosthetic hand, it can be difficult to learn the respective signal patterns. Moreover, it may not be possible, or only possible to a restricted extent, to produce certain signal sequences on account of individual characteristics of the user.

It is therefore an object of the present invention to provide a method and a system for carrying out the method, by means of which there can be a simplified adaptation to the respective user.

According to the invention, this object is achieved by a method having the features of the main claim and a system having the features of the alternative independent claim. Advantageous embodiments and developments of the invention are disclosed in the respective dependent claims, the description, and the figures.

The method for setting up a controller of an orthopedic device comprising at least one motor drive, which is placed against a body part of a patient and connected to sensors that record control signals of the patient, provides for the output of an optical, acoustic, and/or tactile representation of an actuation of a limb as a prompt for the patient to carry out this activity. Subsequently, control signals produced as a deliberate reaction by the patient after being prompted are captured. The captured control signals are assigned to the activity carried out and hence identified as control signals that can serve as switchover signals. Moreover, these captured control signals are assigned to a function, within the scope of which the at least one motor drive is activated, deactivated, or reversed in terms of its direction of rotation such that a link between the captured control signals and the function to be carried out, e.g., “wrist flexion” or “wrist extension”, by the orthopedic device is set up. After the assignment of the captured control signals to the respective function, the captured control signals and/or the function is/are output so that the preferably automated assignment can be captured on account of a standardized default. The output allows checking of the automated calculation of the setting parameters or of the selected function such that an adaptation to the individual care optimum of the user of the orthopedic device is rendered possible. Moreover, feedback is provided for the user and the respective control signals are documented such that statements about an improvement or a deterioration of the respective state can be made over the course of use of the orthopedic device, for example a prosthesis or orthosis. Using the proposed method, the respective control signals are evaluated and checked in respect of their suitability for selecting a corresponding function. This is implemented automatically and so the settings of the parameters such as signal sequence, signal duration, and signal intensity no longer depend on the empirical values of an orthopedic technician but are implemented on the basis of an automated analysis of the control signals instead. Here, the function selection should be considered separately from the implementation of the function. The implementation of the function is triggered by a different signal. The so-called activation signal activates or deactivates the drive in the function assigned thereto in a manner as stored in the controller. If an activation signal is produced by the patient while the first function was selected by the corresponding control signal or the switchover signal, a wrist flexion drive, for example, is activated until the activation signal is no longer produced. If a different function, for example the wrist rotation, is subsequently selected using a switchover signal, the activation signal activates the drive for the wrist rotation for as long as and as quickly as the activation signal is applied. Consequently, the patient need only produce an activation signal which, following the selection by the control signal of the respective function to be carried out, activates or deactivates the respective drive or reverses the direction of rotation of said drive. The function determines what can be carried out, the activation signal determines how the function is carried out, and the control signal switches between the available functions. How the switchover occurs and is setup best is an aspect of the invention.

By way of the output of the control signals to the user or an orthopedic technician, it is possible to highlight possible improvements for the user when producing the control signals and consequently provide feedback as to whether the control signals are even suitable, as to whether the quality of the signals has changed, and as to whether there has been an improvement or deterioration in relation to previous measurements or capture periods.

In a development of the method, provision is made for the control signals to be recorded by way of exactly two electrodes or one electrode pair. In addition to the option of recording at least one control signal, in particular a myoelectric signal, by way of at least one electrode, provision is made for two or more electrode pairs to be present for recording the control signals; then, signal capture is implemented by way of two or more channels when recording the control signals by way of electrode pairs such that switchover signals can easily be produced. The two electrode pairs or sensor pairs produce switchover signals which serve to activate or deactivate the drive or reverse the drive in the direction of becoming active. By capturing the control signals by way of exactly two electrodes or one electrode pair, the complexity of the signal recording is reduced and the production of control signals, in particular the production of myoelectric signals, is simplified since only limited muscle groups need to be activated. In the case of an arrangement at antagonistic muscles, it is possible to obtain a high signal quality of the respective switchover signals, even if two electrode pairs are used.

A development provides for parameters that are relevant to the assignment of the respective function to the control signals to be derived from the captured control signals. By way of example, these parameters are the signal length, the signal location, or the channel on which the signal is produced or recorded, the number of signals, the frequency of signals, and amplitude of signals, particularly if myoelectric signals are captured. In principle, the control signals are signals that are deliberately produced by the person. In addition to myoelectric signals, these can also be pressure signals or signals from pressure sensors or optical signals, or signals correlated to a blood flow.

A development of the invention provides for the prompt to move to be output in the form of suggested switching signals. By way of example, a predefined number or a predefined pattern of muscle contractions can be provided as switching signals, from which the parameters for the control signals are ascertained. By way of example, the switching signals are pulse increase, pulse level, pulse duration of contractions and myoelectric signals derived therefrom.

A development of the method provides for a control signal to be assigned to a function on the basis of a signal strength once a predefined threshold has been exceeded. Setting a threshold prevents control signals being produced by unintended actions, for example involuntary movements or cocontractions, and being considered or interpreted as signals for triggering a function. Only if the captured signal has a certain quality, e.g., a certain intensity, pulse increase, pulse level, or pulse duration, is the latter used as a trigger signal; otherwise, such a signal is not used to initiate a function.

A confirmation can be requested before the assignment of control signals to a function in order to confirm or correct the proposed or automatic assignment of the control signal to a function. The confirmation request is made automatically, a confirmation is then implemented by the orthopedic technician or the user of the orthopedic device.

In one development, the control signals are evaluated in respect of the signal quality before the assignment to a function or a switchover process to another function. The signal quality consists, in particular, in the duration of the signal, the intensity or strength of the signal, and the repeatability. There can be multiple prompts for the patient or user of the orthopedic device to carry out one and the same activity, for example, to straighten a leg or close a hand. If the control signal produced by the patient and captured by the sensors is not output with a sufficient quality or with a sufficient identifiability over all repetitions, sufficient reproducibility is not present, and so there cannot be an assignment of the control signal as a switchover signal to the respective function or as an activation signal. An error message or a correction suggestion is output in the case of an insufficient signal quality, wherein the correction suggestion, as feedback, can also be used as a training request. As a result of the feedback or the correction suggestion, the patient or the user of the orthopedic device is compelled to carry out a multiplicity of, e.g., muscle contractions with unchanging intensity, duration, and/or speed in order to attain the desired signal quality. What this can prevent is a reduction of the muscle activity, for example, over time and a reduction in the contraction ability, contraction strength, and contraction speed, as well as possibly a contraction duration at a certain contraction strength. As a result of the correction suggestion of, e.g., increasing the intensity of the contraction in order to attain the already set signal quality, it is possible to prevent or slow down muscular atrophy.

Predefined or calculated correction factors can be applied to the control signals before the assignment to the respective functions or the switchover procedure to a function. The correction factors can relate to thresholds, times, or gain factors such that the recorded and captured signals are prepared, filtered or electronically altered in any other way so that they are better suited to the identification and triggering of functions.

The control signals produced by the patient as a deliberate reaction following the prompt are stored after every prompt in one development in order to make these control signals available for averaging or to be able to evaluate said control signals over time. On the basis of the stored control signals following every prompt, it is possible to trace a development of responses to prompts, and so, for example, it is possible to capture and trace changes in the pulse height, pulse duration, or pulse increase.

In one development, the control signals produced by the patient as a deliberate reaction following the prompt are compared to predefined target values and assessed in respect of attaining these target values. If the predefined target values are reached, a positive confirmation signal is output, or the attainment is noted in a corresponding file. If a predefined target value is not reached or only just reached, an error message or alert is output in order to inform the patient that the control signal produced does not meet, or almost does not meet, the requirements for triggering a function.

The system for carrying out a method as described above provides for an orthopedic device which is able to be placed against a body part of a patient and comprises at least one motor drive, and comprises an output device which outputs optical, acoustic, and/or tactile representations of an actuation of a limb as a prompt for the patient to carry out this activity, and comprises sensors which are connected to the orthopedic device, are able to be fastened to the patient, and record control signals of the patient. The system furthermore comprises an electronic evaluation device in which the control signals produced by the patient as a deliberate reaction following the prompt are processed, evaluated, and assigned to a function. In particular, the evaluation device is provided as a computer or a data processing device with a memory and possibly a power supply and an output device in order to record, process, store, and output the signals, either to a motor drive or to an output device, which either outputs the sensor signals as such or evaluated and prepared sensor signals. The output device moreover outputs the respective function assigned to the control signal so that it is possible to check and evaluate whether the assignment of the control signal as a switchover signal to the function, undertaken by the evaluation device, is appropriate, the quality of the control signal, whether the function assigned to the control signal is the desired or best suited function or whether a different function is more likely to be suitable for selecting the function in order to operate the orthopedic device therewith.

The evaluation device can comprise an interface, by means of which the assignment of the control signals to the function is able to be influenced so that an automatic assignment undertaken by the evaluation device can be corrected, deleted, or complemented.

Exemplary embodiments of the invention will be described in more detail below on the basis of the attached figures. In detail:

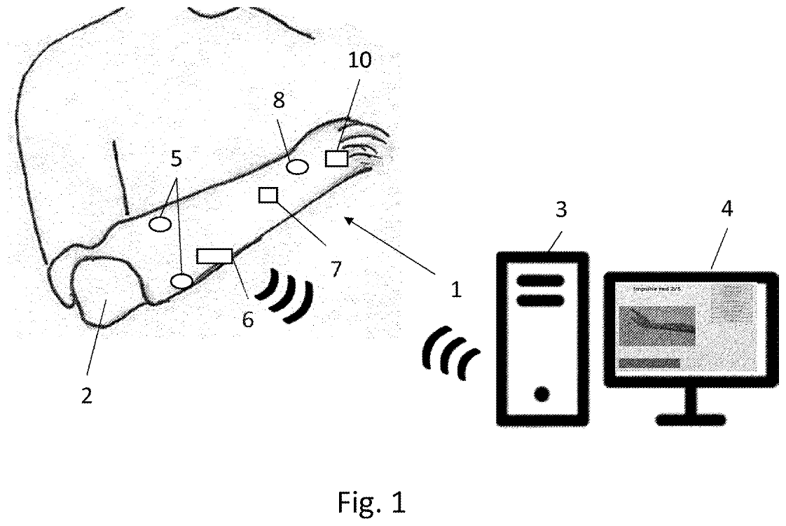

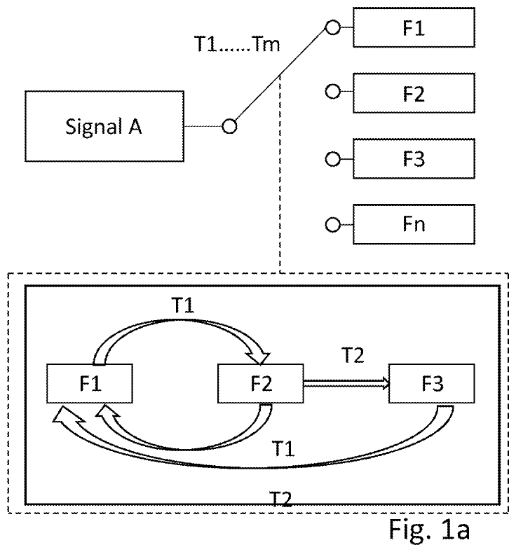

FIG. 1 shows a schematic illustration of a driven orthopedic device, which is connected to a computer;

FIG. 1a shows a schematic illustration of the functionality of switchover signals and activation signals;

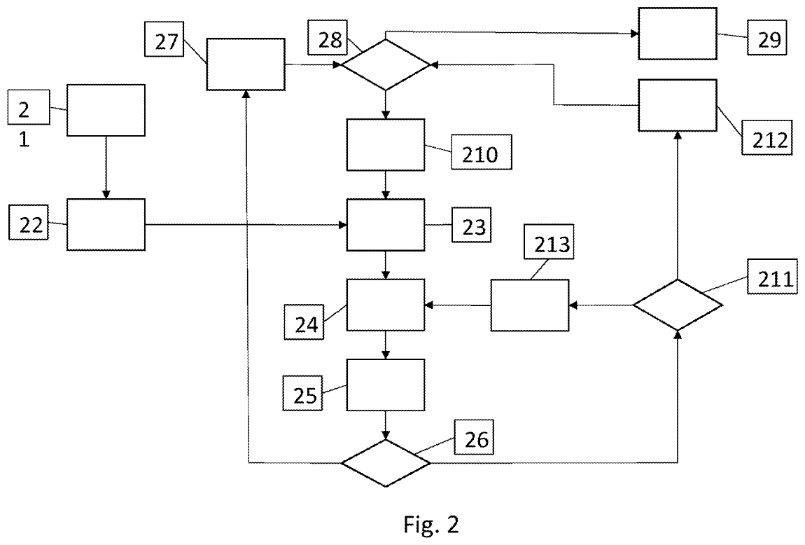

FIG. 2 shows a flowchart;

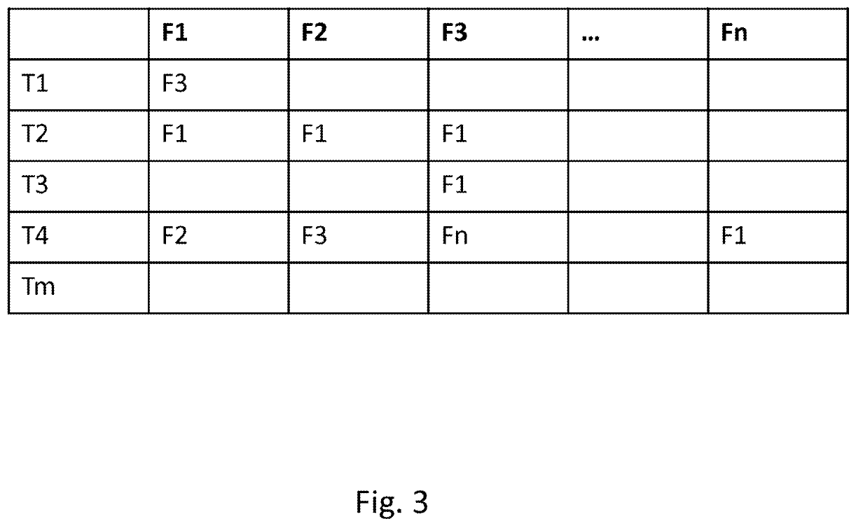

FIG. 3 shows an assignment matrix of possible trigger signals and possible functions;

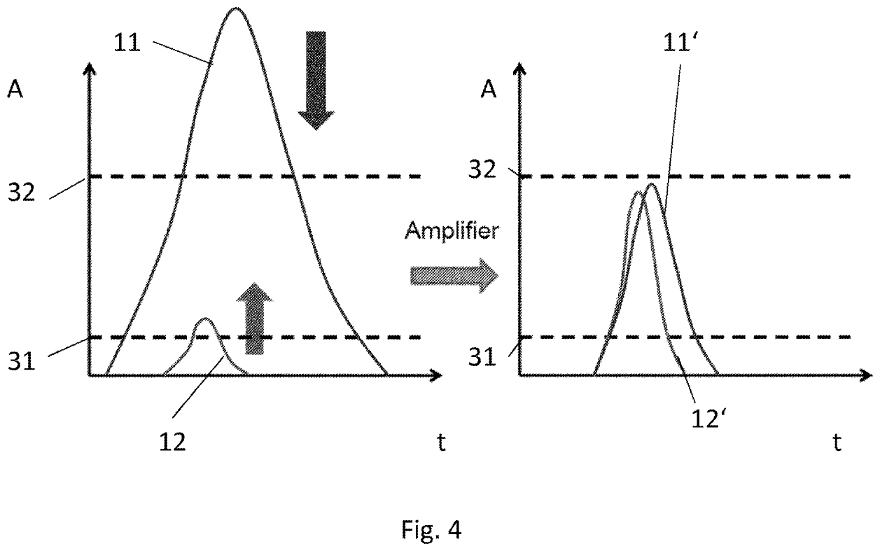

FIG. 4 shows signal representations before and after an amplification; and

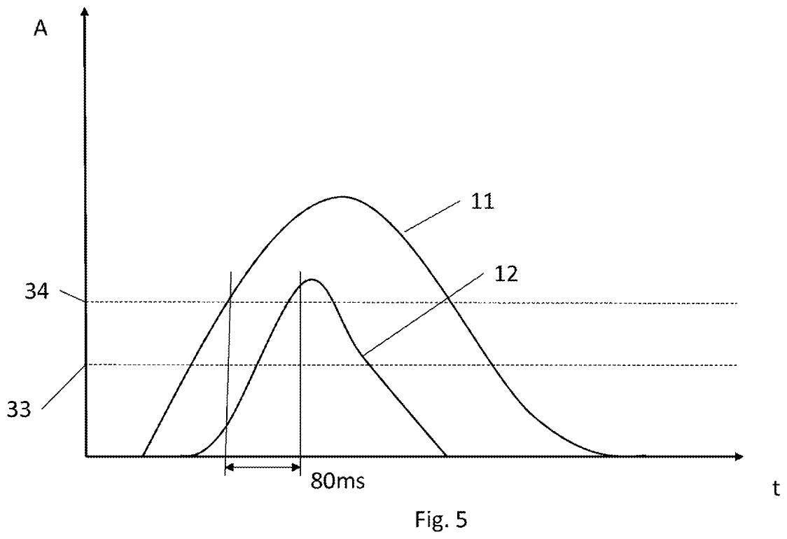

FIGS. 5-8 show conditions in respect of the signal quality.

FIG. 1 illustrates an orthopedic device 1 in the form of a hand prosthesis in a schematic illustration. The orthopedic device 1 comprises a forearm socket, which is secured to a forearm stump on an arm 2 of a patient. As an alternative to a prosthesis, the orthopedic device 1 can also be embodied as an orthosis. As an alternative to an orthopedic device on an upper extremity, it can also be secured to a lower extremity, for example as a leg orthosis or a prosthetic leg. Sensors that capture control signals produced by the patient or the user of the orthopedic device 1 are arranged on the orthopedic device 1. Said control signals can be contraction signals in particular, i.e., signals based on muscle contractions. In addition to myoelectric signals in particular, these can also be direct nerve pulses, density changes, temperature changes, flow resistances, or electrical resistances.

At least one motor drive 10, in particular an electric motor drive, is provided in the orthopedic device 1 and able to be controlled by a control device 7 in order to move components of the orthopedic device 1, for example to move individual fingers, in order to rotate the prosthetic hand about the stump longitudinal axis, in order to carry out a wrist flexion or a wrist extension, or in order to be able to carry out other movements of a motor driven prosthetic hand. In accordance with the structure of the orthopedic device, the drives 10 can be controlled separately from one another or else they can be controlled together. In another embodiment of the orthopedic device 1, for example as a leg orthosis, the drives 10 can bring about a flexion or extension of a shank part or a foot part.

The orthopedic device 1 likewise comprises a power storage device 8 for supplying the motor drive 10 with sufficient power, for example with electric power. The respective functions or switching or switchover processes for the respective motor drive 10 are coordinated and brought about by the control device 7. The control device is connected to the sensors 5, for example to an electrode pair, by means of which the control signals are recorded on account of cocontractions in the forearm. The orthopedic device 1 likewise has assigned a transmitter and receiver device 6, which allows control signals, prepared control signals, or other information items to be received or transmitted. The recorded control signals can be sent to a computer 3 for example, in which the recorded control signals are processed. The computer 3 is connected to an output device 4, which is a display in the illustrated exemplary embodiment. The output device 4 can also output an acoustic representation and/or a tactile representation in addition to an optical representation, for example by way of low frequency vibrations or else by movements of models.

To fit the orthopedic device 1 to the respective patient, it is necessary to record certain control signals, which are captured by the sensors 5 and initiated by the patient, and assign these to certain functions which are stored in the control device 7 and linked to switching processes or switchover processes for the drives 10. By way of example, as switchover signal 1, switchover signal 2, switchover signal 3, etc., a certain contraction pattern is assigned in the process to closing of the hand, another is assigned to opening, a third is assigned to a wrist rotation, a further pattern is assigned to wrist flexion, extension, etc. The contraction patterns are not converted directly into movements but serve as switchover signals in order to arrive at a function B from a function A. A conventional approach until now has been that a patient had to practice predefined contraction patterns until the control signals produced thereby correspond to the values predefined in the control device 7. Particularly in the case of conventional care with one electrode pair, i.e., with two channels, this is extremely difficult and strenuous for the patient. A multiplicity of contraction patterns must be able to be produced in ongoing fashion and stably, and the contractions need to be applied in the correct sequence, with the right strength and over the right amount of time. The switchover from one function to another function, introduced by way of a further contraction pattern, for example, is particularly difficult. What is achieved by the present invention is that, firstly, switchover signals that the user cannot carry out, for example on account of injury, are eliminated and that, secondly, switchover signals which can be carried out by the user are optimized in terms of their parameters in such a way that the user can carry said switchover signals out easily and repeatedly. The selection of the parameters and the optimization of the parameters are preferably implemented automatically and not on the basis of a non-reproducible feeling or the experience of an orthopedic technician.

In order to make the use of an orthopedic device 1 easier for a patient, in order to improve the fit of the respective orthopedic device 1 to the patient and, in particular, in order to simplify the setting up method, an optical, acoustic, or tactile representation of an actuation of a limb is output to the patient via the output device 4 as a prompt to carry out this activity. There should be a wrist extension in the illustrated exemplary embodiment. After perceiving the output in the output device 4, the patient carries out this voluntary activity. In the case of prosthetic care, naturally, the movement itself cannot be carried out and only the muscles that the patient would naturally use to this end are activated; thus, there this is a corresponding contraction of the muscles involved for this movement. In the case of orthotic care, the movement might be able to be carried out but not with a sufficient strength, and so assistance by a drive 10 is necessary.

Control signals, for example myoelectric signals on account of muscle contraction, are captured via the sensors 5 or the electrode pair. The captured control signals are assigned to the carried-out activity, preferably assigned automatically, and transmitted to the computer 3, preferably in wireless fashion, via the transmitter and receiver device 6. Alternatively, a wired link is provided between the orthopedic device 1 and the computer 3. In the computer 3, the captured control signals are assigned to the demonstrated actuation of a function shown on the output device 4, within the scope of which function the at least one motor drive 10 is activated, deactivated, or reversed in terms of its direction of rotation. The control signal pattern assigned to the wrist extension is assigned to the function and converted into corresponding switching commands for the respectively required motor drive 10 in order to be able to carry out a wrist extension of the orthopedic device 1.

FIG. 1a illustrates the basic difference between the switchover between individual functions and the activation of the respective function. The upper illustration shows signal A to the left, which signal activates the respectively selected or switched function. The individual functions F1, F2 to Fn are illustrated to the right in the upper illustration. By way of example, the functions F1, F2, F3 to Fn set what movement can be carried out, for example an internal rotation, an external rotation, closing of a hand, opening of a hand, the extension of a forearm or flexion. In order to be able to switchover between the individual functions F1 to Fn so that an external rotation can be implemented after an internal rotation of a prosthetic hand or else so that something can be gripped by closing a prosthetic hand after a forearm extension, the control signals are assigned to a function and serve as switchover signals or trigger signals which make it possible to arrive at the function F2 or F3 from the function F1 so that a different type of movement can even be carried out. The control signals as switchover signals or trigger signals do not trigger an actuation of the orthopedic device or an activation or deactivation of a motor drive but only specify which function can be carried out by the patient with a subsequent signal, a so-called activation signal. By way of example, using the control signal in the form of the trigger signal or switchover signal T1, the function F1 with the possibility of closing a prosthetic hand is selected. This function F1 is stored with the commands relating to which motors or which motor are/is activated in which direction of rotation as soon as an activation signal is received. A trigger signal T2 can select the function F2 with the action of opening the hand, for the purposes of which the drive direction of the motor or the motors is reversed. The remaining functions F3 to Fn can be correspondingly stored with actions and can be selected by the corresponding trigger signal. Muscle contractions, pulse sequences, pulse durations and the like can be used as control signals or trigger signals. Once the respective function has been selected, the action or movement connected therewith is activated by the activation signal A. This signal A activates the respective switched function and activates or deactivates the drive or reverses the direction of rotation, wherein this signal A can also serve for proportional control of the respective drive. By way of example, if an increasing contraction is determined, the action can be carried out at a higher speed or with a greater force, for example in order to grip an article faster or more securely.

The lower illustration of FIG. 1a illustrates the switchover between the individual functions F1, F2, and F3 in exemplary fashion. If the function F1 is currently set, there can be a switch to the second function F2 by the control signal as trigger signal T1. There is a switch back to the initial function F1 by carrying out the control signal T1 again. If, when proceeding from the function F2, a second, different control signal T2 is produced by the patient, there is a switch to the next function F3. There is a switch back to the function F1 again by way of a renewed trigger signal T2. Alternative switchover rules may be present; for example, there can be a switch back to the function F2 by a renewed trigger signal T2, from where there can be a switch to function F1 by producing the trigger signal T1 again.

Before there is a final assignment of the captured control signals to the respective function, the captured control signals are output by way of the output device. As an alternative or in addition thereto, the respective function can be output after the respective assignment of the control signal as trigger or switchover signal for this function in order to check whether the assignment is advantageous or expedient. By way of this output, there can be feedback in respect of the current activation status and the respective assignment of a control signal or of a plurality of control signals to a respective function. Since the assignment was initially proposed in automated fashion within the scope of a standard program, there can be an individual adaptation, for example by an orthopedic technician, using the output. This will simplify the setting up process of orthopedic devices, in particular of myoprostheses, since there is an automated calculation of the setting parameters which can additionally be fitted to the individual care optimum of the respectively cared-for person. Additionally, by way of the feedback, there can be in the output device 4 a documentation of the control signals and a recommendation in respect of an improved use of the orthopedic device or, possibly, training advice for the user.

In the computer 3, the desired parameters for the respective function are calculated from the control signals 5. Here, the switchover signals or control signals that are possible for and actually carried out by the patient or user in each case are identified and the optimized parameters are defined for the respective function. Thus, the possible control signal for the user, which can be provided by them, is captured and evaluated and mapped to the necessary or possible functions of the orthopedic device 1 such that a parameter setting for the switchover processes between the available or possible functions can be proposed and can be adapted on an individual basis following the output in the output device 4. Thus, thresholds, time windows, or gain factors of the signals, for example, can be selected and set automatically.

FIG. 2 shows a possible course of events for a method for setting a control device for an orthopedic device, which includes a first step 21. A user is identified in the first step 21, and user data of the user are recorded and stored and assigned to the respective process where necessary. Moreover, instructions are made available to the user by a recording process for collecting all relevant movement patterns. By way of example, the totality of all possible functions that the orthopedic device can carry out are stored on the computer. In order to be able to assign these functions to the respective control signals of the patient, the respective functions are queried via the output device 4 in individual program steps. As it were, the user must then carry out the corresponding movements, at least want to deliberately carry out the muscle contractions or movements, so that control signals can then be captured by way of the sensors 5. Since these control signals must have a certain quality, gain factors can be applied thereto, by means of which the signal strength can be increased or decreased. Moreover, a check must be carried out as to whether the captured control signals lie in a parameter range which only makes an evaluation possible. As a rule, the individual programs are run through multiple times in order to be able to ensure a sufficiently high repetition accuracy.

In the second step 22, the first program is started after the introduction. Initially, the greatest possible gain factor for the control signals is chosen by the sensors 5 in the step 23.

In step 24, the collected user data are amplified by the currently selected, greatest possible gain factor. Subsequently, all relevant parameter values for the currently carried out program are calculated for the amplified user data in step 25; by way of example, these are the signal duration, the signal amplitude, the attainment of thresholds, or thresholds being exceeded.

In the next step 26, there is a query as to whether all relevant parameters, which are required for the respective function, lie in the value range respectively admissible therefor. If this is the case, the program is stored as functional in a step 27 and the current gain factor is stored as an associated, optimum gain factor. After being stored as optimum gain factor, a check is carried out in the next step 28 as to whether the currently current program was the last program to be checked, i.e., whether all possible functions for setting this orthopedic device 1 for this patient have been queried. If this is the case, a list of all functional programs is provided in the step 29 for provision on an output device 4 and, where necessary, for checking by an orthopedic technician or any other person. In this list or in the representation of all functional programs, it is possible to select the respective functions and modify these later where necessary.

If the query in step 28 yields that the current program was not the last program to be checked, the next program in the list of all possible programs is selected in step 210 and this program is supplied to work step 23 such that the next steps run as described above.

If it is determined in work step 26 that not all relevant parameters lie in an admissible range, there is a query in a step 211 as to whether the current gain factor was the smallest possible gain factor. If the reply to this query is in the affirmative, i.e., if there is no smaller gain factor, the program carried out is stored as nonfunctional in a step 212. Thus, the signal strength, signal fidelity, rhythm, edge steepness or other parameters attained by the patient are therefore not sufficient for generating a sufficiently clear trigger signal so that the function can be carried out. Provided the nonfunctional program was the last program to be checked, a list with all functional programs is generated in step 29 according to step 28. If the current program is not the last program to be checked, the next program in the list is chosen as per step 211 and steps 23 to 26 are run through.

If a reduction in the gain factor was possible in method step 211 when there was a query as to whether the current gain factor was the smallest, i.e., if the current gain factor was not the smallest possible gain factor, the next smaller gain factor is chosen in step 213 and the program procedure is carried out again with step 24, specifically by virtue of the user data with the currently chosen, i.e., the next smallest gain factor being amplified and, subsequently, the relevant parameters being calculated for the current program with the user data amplified thus.

FIG. 3 shows the result of a complete program run through with all possible functions of the orthopedic device 1, in which the trigger signals or control signals T are plotted in the left-hand column and the functions F are plotted along the first line. Trigger signals T1 to Tm are possible, and functions F1 to Fn are available as functions. Examples of trigger signals with sensors 5 as two electrode pairs for providing two-channel care would be, for example, a short signal sent simultaneously on two channels, a long signal set simultaneously on two channels, a short pulse on the first channel, a short pulse on the second channel, a time switch or a fast signal, which is usable, in particular, in the case of four-channel control. The opposition grip, the lateral grip, a hook function, a wrist flexion, a wrist extension or a wrist rotation serve as examples of functions F of an orthopedic device 1 in the form of a hand prosthesis.

Different functions F1 to Fn can be attained with different trigger signals T1 to Tm. The completed assignment after one program run through is shown on the basis of the exemplary switching matrix. From the matrix, it is possible to gather what function F can be obtained or introduced after the introduction of a trigger signal T when a certain function F is used as a starting point. By way of example, function F3 is reached if function F1 is active and the trigger signal T1 is carried out.

Function F1 is reached if function F3 is active and trigger signal T2 is carried out. Independently of the currently active function, there always is a return to function F1 by activating the trigger signal T2. Carrying out the trigger signal T4 always switches one function further, i.e., function F2 is reached starting from function F1 by activating the trigger signal T4, function F3 is reached starting from the function F2 after carrying out the trigger signal T4, etc. Consequently, it is possible to switch through all functions in sequence using the trigger signal T4.

Once such a matrix has been created after the control signals have been evaluated, an orthopedic technician, for example, can identify which function of the prosthesis is only able to be controlled by the specific patient in order to then carry out said function using a separate activation signal. The patient can also gather from the matrix which control signal or trigger signal is best suited to actuate and subsequently activate the functions available.

In the left-hand illustration, FIG. 4 shows a two-channel control signal of a cocontraction with a first signal 11 and a second signal 12. The first signal 11 has a substantially greater amplitude A and a longer signal duration T than the second signal 12. The first signal 11 exceeds an upper limit value 32 while the second signal 12 exceeds a lower limit value 31. Following the request directed to the patient to carry out a certain movement, a longer and stronger contraction of the muscle arises in the example of FIG. 4, leading to the first signal 11, while a cocontracted muscle is tensed for a shorter time and less strongly such that the second signal 12, a myoelectric signal, is correspondingly shorter and has a lower amplitude. To be able to reliably provide a trigger signal T or switchover signal for switching over to another function for the control unit 7, both signals 11, 12 should lie between the two thresholds 31, 32. During the signal evaluation, there is a detection by way of an algorithm that the first signal 11 needs to be damped, i.e., needs to be provided with a negative gain factor, while the second signal 12 needs to be amplified, i.e., needs to have a positive gain factor applied. Both signals 11, 12 should lie is close as possible to the upper threshold 32 in order to ensure a sufficient signal strength and hence a sufficient identifiability. Accordingly, the first signal 11 is damped and compressed in respect of the time duration by way of an amplifier or an amplification function while the second signal 12 is amplified in respect of the amplitude. The time duration of the second signal 12 remains unchanged, but the first signal 11 is shifted in respect of the start to the start of the second signal 12. The result is shown in the right-hand illustration of FIG. 4, in which the optimized signals 11′, 12′ still have their characteristic form but are modified in terms of their amplitude and their duration. During the amplification, care must be taken that all other trigger signals or control signals likewise work such that a common gain factor can be applied in respect of the amplitude and the time shift and time reduction in respect of the time duration of the signal can be applied uniformly to all control signals.

FIG. 5 shows an exemplary condition that must be satisfied so that it is even possible to evaluate and amplify a two-channel control signal with a first signal 11 and a second signal 12. As a condition, provision is made for both signals 11, 12 to have to exceed an activation limit value 34 within a certain time period. The activation limit value 34 relates to the signal amplitude; in the illustrated exemplary embodiment, the first signal 11 reaches the activation threshold 34, which is above a deactivation threshold 33, first and said activation threshold is then also attained by the second signal 12 within a time period of approximately 50 ms. The second signal 12 reaches the activation limit value 34 within the specified time period, 80 ms in the illustrated exemplary embodiment, and so the first condition for the evaluation of the recorded signal is satisfied and both signals 11, 12 have an initially sufficient signal quality.

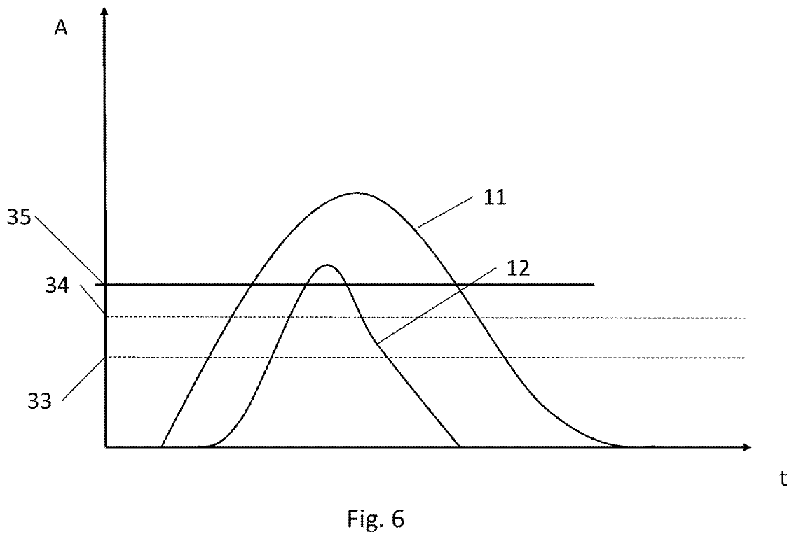

FIG. 6 shows a further requirement on both signals 11, 12 for these to be able to serve as a trigger signal T for introducing or switching over between individual functions F. Both signals 11, 12 must exceed a cocontraction threshold 35, i.e., reach a certain minimum amplitude which is above the activation limit value 34. By way of example, if the activation limit value 34 is 0.5 V and the deactivation limit value is 0.3 V, the cocontraction threshold can be 0.6 V.

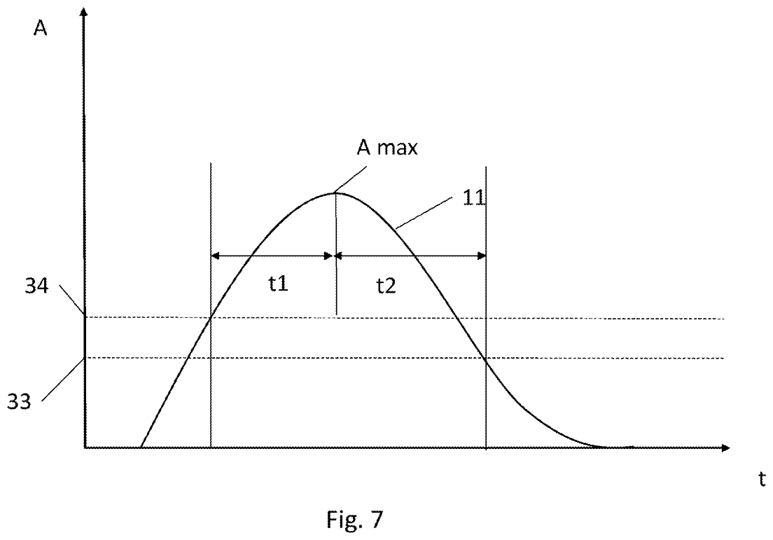

A third condition for a sufficient signal quality is illustrated in FIG. 7, according to which the amplitude must be above one of the two limit values 33, 34 during a predetermined time period T1 before the maximum amplitude value Amax is reached and during a second time period T2 after the maximum amplitude Amax was reached. By way of example, a stipulation can be that a maximum amplitude value Amax must be reached within a certain time period T1 after passing through the activation limit value 34 and the signal amplitude must be above the deactivation limit value 33 during a second time period T2 after the maximum amplitude value Amax has been reached so that the cocontraction signal, in the present case the first signal 11, can be used.

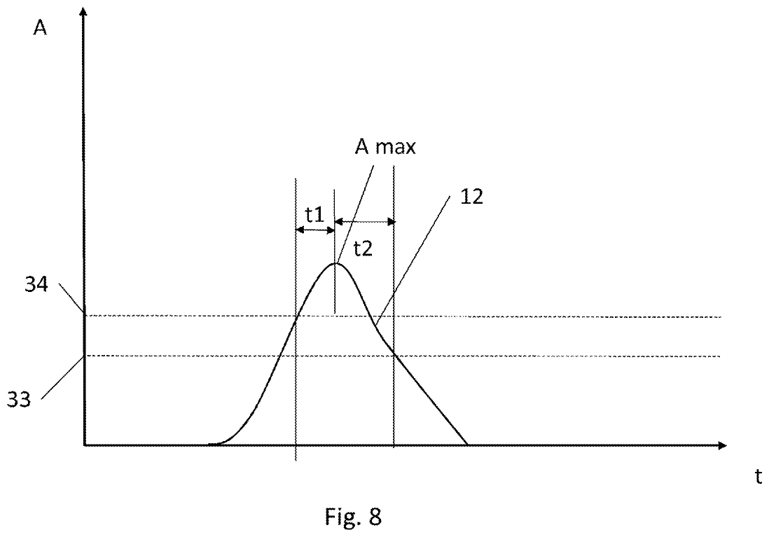

FIG. 8 shows the corresponding condition for the second signal 12, which is captured by a second electrode, while the first signal 11 is captured by a first electrode or a first sensor 5.

If all conditions are satisfied, the received control signals can be prepared in the computer 3 by way of the respectively chosen gain factor, and so both the captured control signals and the gains and the assigned functions F can be displayed by way of the output device 4. The gain factors can be subsequently adapted, for example by way of a user interface on the output device 4 of the computer 3. By way of the control signals captured after the output of the movement prompt, it is possible to capture and assess the signals the respective patient is even capable of in relation to their signal quality, and assign said signals to the corresponding movements. If the patient is unable to produce a sufficient signal quality for the switchover to a specific function or for a specific predefined sequence of movements, this function can either be omitted and stored in the control device 7 as not activated, or else a different signal profile becomes necessary or else a different gain factor is chosen.

The output of the control signals produced by the patient moreover serves documentation purposes in respect of which control signals the patient is even capable of. If there is a drop in signal quality, for example if a patient can no longer attain a certain signal strength or amplitude A, this may serve as an indicator to prescribe a training program or else to document training progress.

1. A method for setting up a controller of an orthopedic device comprising at least one motor drive, which is placed against a body part of a patient and connected to sensors that record control signals of the patient, including the following steps:
outputting an optical, acoustic, and/or tactile representation of an actuation of a limb as a prompt for the patient to carry out this activity;
capturing control signals produced by the patient as a deliberate reaction by the patient after being prompted,
assigning the captured control signals to the activity carried out and to a function, within the scope of which the at least one motor drive is activated, deactivated, or reversed in terms of its direction of rotation, and
outputting the captured control signals and/or the function after the assignment to the respective function.

2. The method of claim 1 wherein the control signals are recorded by way of exactly two electrodes or one electrode pair.

3. The method of claim 1, wherein parameters that are relevant to the assignment of the respective function are derived from the captured control signals.

4. The method of claim 1, wherein the prompt is output in the form of predefined switching signals.

5. The method of claim 1, wherein a control signal is assigned to a function on the basis of a signal strength once a predefinable threshold has been exceeded.

6. The method of claim 1, wherein a confirmation is requested before the assignment of a captured control signal to a function.

7. The method of claim 1, wherein the control signals are evaluated with respect to the signal quality before the assignment to a function and wherein an error message or a correction suggestion is output in the case of an insufficient signal quality.

8. The method of claim 1, wherein predefined correction factors are applied to the control signals before assignment.

9. The method of claim 1, wherein the control signals produced by the patient as a deliberate reaction following the prompt are stored after every prompt.

10. The method as claimed in claim 1, wherein the control signals produced by the patient as a deliberate reaction following the prompt are compared to predefined target values and assessed for their relationship to these target values.

11. A system for carrying out the method of claim 1, comprising
a. an orthopedic device which is able to be placed against a body part of a patient and which comprises at least one motor drive, the system further comprising:
b. an output device which outputs optical, acoustic and/or tactile representations of an actuation of a limb as a prompt for the patient to carry out the represented actuation of a limb,
c. sensors which are connected to the orthopedic device, able to be fastened to the patient, and to record control signals produced by the patient,
d. an electronic evaluation device in which the control signals produced by the patient as a deliberate reaction following the prompt are processed, evaluated, and assigned to a function, and
e. an output device, in which the respective function assigned to the control signal is output.

12. The system of claim 11, wherein the evaluation device comprises an interface, by means of which the assignment to the function is able to be influenced by a user.

13. A method for setting up a controller of an orthopedic device comprising at least one motor drive, the method including the steps of:
placing the orthopedic device against a body part of a patient;
connecting the orthopedic device to two electrodes or an electrode pair that record control signals produced by the patient;
outputting an optical, acoustic, and/or tactile representation of a limb actuation as a prompt for the patient to carry out the represented limb actuation;
capturing control signals produced by the patient after the representation is output with the two electrodes or electrode pair;
assigning the captured control signals to the activity carried out and to a function, within the scope of which the at least one motor drive of the orthopedic device is activated, deactivated, or reversed in terms of its direction of rotation;
requesting confirmation of the assignment of the captured control signal to a function; and
outputting the captured control signals and/or the function after the assignment to the respective function.

14. The method of claim 13 wherein control signal parameters that are relevant to the assignment of the function to be assigned are derived from the captured control signals.

15. The method of claim 13, wherein the prompt is output in the form of predefined switching signals.

16. The method of claim 13, wherein a control signal is assigned to a function on the basis of a signal strength once a predefinable threshold has been exceeded.

17. The method of claim 13, wherein the control signals are evaluated with respect to the signal quality before the assignment to a function and wherein an error message or a correction suggestion is output in the case of an insufficient signal quality.

18. The method of claim 13, wherein predefined correction factors are applied to the control signals before assignment to a function.

19. The method of claim 13, wherein the control signals produced by the patient as a deliberate reaction following the prompt are stored after every prompt.

20. A method for setting up a controller of an orthopedic device comprising at least one motor drive, the method including the steps of:
placing the orthopedic device against a body part of a patient;
connecting the orthopedic device to two electrodes or an electrode pair that record control signals produced by the patient;
outputting an optical, acoustic, and/or tactile representation of a limb actuation as a prompt for the patient to carry out the represented limb actuation;
capturing control signals produced by the patient after the representation is output with the two electrodes or electrode pair;
storing the control signals produced by the patient after the representation is output;
comparing the control signals produced by the patient to predefined target values and assessing the control signals for their relationship to the target values
assigning the captured control signals to the activity carried out and to a function, within the scope of which the at least one motor drive of the orthopedic device is activated, deactivated, or reversed in terms of its direction of rotation;
requesting confirmation of the assignment of the captured control signal to a function; and
outputting the captured control signals and/or the function after the assignment to the respective function.
